(19) United States
(12) Patent Application Publication
(10) Pub. No.: US 2008/0249563 A1
(43) Pub. Date: Oct. 9, 2008

(54) Method and apparatus for tissue fastening

(76) Inventors: James A. Peterson, Edina, MN (US); David B. Herridge, Mendota Heights, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/732,704

(22) Filed: Apr. 4, 2007

(51) Int. Cl. A61B 17/03

(52) U.S. Cl. 606/216

(57) ABSTRACT

Methods and apparatus directed to tissue capture, presentation and retention using a surgical apparatus. One or more tissue capture arms can include surface features that engage captured tissue and provide omni-directional support during capture and retention of the tissue. The tissue clamping arm can include improved centering features for positioning captured tissue relative to a penetrator that is advance into the capture tissue. Through improved tissue handling and tissue retention relative to the penetrator, captured tissue can be consistently pierced regardless of external forces and techniques. The tissue clamping arm can include gripping members arranged along an interface surface to improve tissue grip and presentation.

### FIELD OF THE INVENTION

The present invention relates generally to the field of surgical instruments such as surgical staplers, clip applicators and sutureless closure devices. More particularly, the present invention relates to improvements in tissue manipulation, retention and presentation during tissue fastening.

### BACKGROUND OF THE INVENTION

When an opening in tissue is created either through an intentional incision or an accidental wound or laceration, biological healing of the opening commences through the proximity of the opposed living tissue surfaces. If the opening is very large or if its location subjects the wound to continual movement, a physician will seek to forcibly hold the sides of the opening in close proximity so as to promote the healing process. Representative methods for forcibly fastening and retaining tissue during healing has included the use of sutures, clips and staples.

In the case of skin tissue, for example, healing occurs best when the opposing dermal layers of the skin tissue are held in proximity with each other. Human skin tissue is comprised of three distinct layers of tissue. The epidermal layer, also known as the epidermis, is the outermost layer and includes non-living tissue cells. The dermal layer, or dermis, is the middle layer directly below the epidermal layer and comprises the living tissue of the skin that is the strongest of the three layers. The subcutaneous, or hypodermis layer is the bottom layer of skin tissue and includes less connective tissue making this the weakest layer of skin tissue.

A recent advance in the area of tissue fastening is the award winning INSORB® Subcuticular Skin Stapler (“the INSORB® staple”) commercially available from Incisive Surgical, Inc. of Plymouth, Minn. (www.insorb.com). A number of patent publications address the technology of the INSORB and include U.S. Pat. No. 6,726,705, as well as in U.S. Publ. Nos. US2003-0236551 A1, US2004-0059377 A1 and US2004-0059378 A1 to Peterson et al, all of which are commonly assigned to the assignee of the present application and all of which are incorporated by reference in their entirety.

In a representative embodiment, the INSORB stapler utilizes a bilateral approach to tissue fastening wherein a fastening apparatus manipulates opposed sides of tissue to form target tissue zones within each tissue side wherein a bioabsorbable fastener is subsequently deployed in a substantially simultaneous bilateral manner to retain opposed sides of tissue in close approximation so as to facilitate tissue healing. By maintaining contact of the tissue throughout the healing process, the healing process is enhanced which results in less chance of infection, faster recovery and improved aesthetic appearance. In addition, no subsequent medical follow-up is necessary to remove fasteners as is typically necessary with non-absorbable fasteners.

While the tissue fastening advantages of the INSORB stapler have been recognized as evidenced by numerous design awards and medical studies, there exists with all varieties of tissue fastening instruments to further improve on the handling and maintenance of tissue as fasteners are introduced.

### SUMMARY OF THE INVENTION

The present invention is directed to improvements in the area of tissue maintenance such as, for example, tissue capture, tissue presentation and tissue retention, during tissue fastening with a tissue fastening apparatus. In one representative embodiment, the tissue fastening apparatus comprises a bilateral subcuticular skin stapler using bioabsorbable staples, such as the INSORB stapler, although the improvements disclosed herein apply equally to other tissue fastening instruments in which tissue maintenance during piercing and placement of a tissue fastener is important for successful tissue capture.

In one aspect, the present invention is directed to tissue capture arms, and more specifically, surface features on said tissue capture arms that engage captured tissue during capture and retention of the tissue. In one representative embodiment, the surface features can comprise a plurality of inwardly projecting “castles” or cubes arranged so as to maximize traction and tissue control throughout a fastener insertion process. The inwardly projecting castles and cubes can define an arcuate profile corresponding to at least a portion of a penetrator radius such that captured tissue is evenly deformed around at least that portion of the penetrator, radius, thereby providing for more consistent fastener placement along an incision. The surface features assist in isolating captured tissue from external forces such as operator fatigue or ergonomic issues that can affect consistency along the length of a wound closure.

In another aspect, the present invention is directed to tissue capture arms, and more specifically, improved centering features on said tissue clamping arms for centering captured tissue and presenting said tissue to one or more penetrators during a fastener insertion process. In one representative embodiment, the improved centering features can comprise a plurality of high and low profile surface projections on an inner surface of each tissue capture arm arranged to retain and position captured tissue as the one or more tissue penetrators are deployed into the captured tissue during fastener deployment. In one embodiment, the high and low profile surface projections can comprise a castle arrangement providing omni-directional support during tissue capture and retention as well as for proving fast tissue decompression upon release of the captured tissue.

In another aspect, the present invention is directed to a tissue capture arm that can capture and temporarily deform tissue to form a tissue radius that generally, evenly surrounds at least a portion of a penetrator radius. Through the matching of a tissue contour with a penetrator contour, a more consistent cut can be made through the tissue, and consequently a more consistent fastener placement can be accomplished without regard to differing techniques of medical professionals or tissue variations commonly found with large incisions such as, for example, an abdominoplasty. Matching the tissue contour with the penetrator contour allows tissue to be controlled essentially equally around the penetrator resulting in increased staple-to-staple consistency along a single closure.

In another aspect, a method for reducing user fatigue during closure of large incisions can by accomplished by providing a fastening instrument having a tissue capture assembly capable of providing omni-directional support during capture and retention of the tissue. The method can further comprise temporarily deforming tissue such that a tissue radius corresponds to a penetrator radius, thereby providing for increased staple-to-staple placement consistency along an incision.

The above summary of the various aspects of the disclosure is not intended to describe each illustrated embodiment or every implementation of the invention. The figures in the detailed description that follow more particularly exemplify these embodiments.

### BRIEF DESCRIPTION OF THE DRAWINGS

These, as well as other objects and advantages of this invention, will be more completely understood and appreciated by referring to the following more detailed description of exemplary embodiments of the invention in conjunction with the accompanying drawings, of which:

FIG. 1 is a perspective view of an opening in skin tissue.

FIG. 2 is a section view of the opening in skin tissue of FIG. 1 taken at line 2-2 of FIG. 1.

FIG. 3 is a section view of the opening in skin tissue of FIG. 1 arranged in a everted disposition.

FIG. 4 is a perspective view of a surgical fastening apparatus in a non-capture disposition according to an embodiment of the present disclosure.

FIG. 5 is a bottom view of a tissue interface portion of the surgical fastening apparatus of FIG. 4 in a capture disposition.

FIG. 6 is a perspective view of a capture lobe and a penetrator with the surgical fastening apparatus of FIG. 4 in the capture disposition of FIG. 5.

FIG. 7 is a section view of a capture lobe and a penetrator taken at line 7-7 of FIG. 8.

FIG. 7a is an end view of a capture lobe according to an embodiment of the present disclosure.

FIG. 7b is an end view of a capture lobe according to an embodiment of the present disclosure.

FIG. 7c is an end view of a capture lobe according to an embodiment of the present disclosure.

FIG. 8 is a bottom view of the tissue interface portion of FIG. 5 including one side of captured tissue.

FIG. 9 is bottom, partially hidden view of the tissue interface portion of FIG. 5 including one side of captured tissue.

FIG. 10 is a section view of a capture lobe and penetrator taken at line 10-10 of FIG. 9.

FIG. 10a is section view of the capture lobe and penetrator taken at an orientation similar to FIG. 10

FIG. 10b is a section view of a prior art capture lobe and penetrator taken at an orientation similar to FIG. 10.

FIG. 11 is a bottom, partially hidden view of the tissue interface portion of FIG. 5 including one side of pierced tissue.

FIG. 12 is a bottom, partially hidden view of the tissue interface portion of FIG. 5 with a surgical fastener place within one side of tissue.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE DRAWINGS

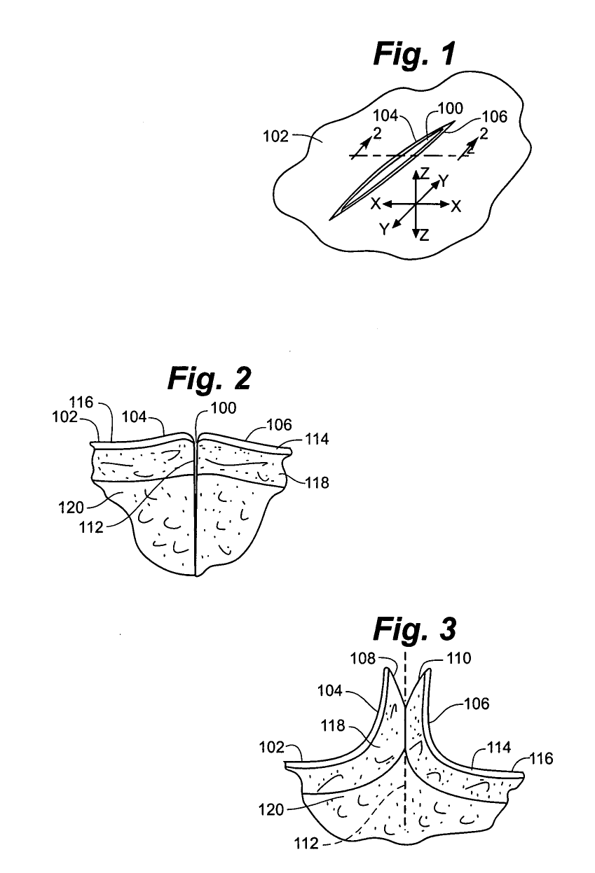

In FIGS. 1 and 2, there is shown a depiction of a typical opening 100 in the surface of skin 102, such as may be made, for example, by a surgical incision or a wound. Opening 100 defines a first side 104 and a second side 106 on opposed sides of the opening 100. First side 104 and second side 106 can comprise a substantially parallel arrangement as illustrated in FIG. 1, or alternatively, first side 104 and second side 106 comprise substantially non-parallel portions such as commonly associated with wounds as opposed to surgical incisions. As illustrated in FIG. 1, for purposes of describing the present invention, opening 100 may be described as having a length or longitudinal orientation parallel to the y-y axis, a width orientation parallel to the x-x axis, and a depth orientation parallel to the z-z axis. The x-y-z axis, for purposes of the present invention, is defined with respect to an external tissue surface, which in the case of skin 102 is the outer surface. References to a vertical and horizontal planar orientation in connection with the present invention are made with respect to the external tissue surface at the site of the opening in question. As described herein, opening 100 can have a length from about 8 mm, which can reflect surgical procedures such as, for example, a mole resection, to about 60 cm, which can reflect surgical procedures such as, for example, an abdominoplasty.

As illustrated in FIG. 3, a first vertical inner surface 108 associated with first side 104 and a second vertical inner surface 110 associated with second side 106 can be visualized as meeting along a generally vertical interface 112. It will be understood that in the case of an opening that extends over a curved tissue surface, the corresponding horizontal and vertical surfaces associated with the opening will be defined with respect to such curved tissue surface.

It also will be understood that the vertical interface 112 may be vertical in only one orientation with respect to the tissue surface, such as in the case when an angled incision has formed the opening 100.

As illustrated in the sectional view of FIGS. 2 and 3, human skin 102 generally has three discrete layers. These layers comprise an epidermal layer 114 of mostly non-living tissue having an exterior surface 116, a dermal layer 118 of mostly living tissue, and a subcutaneous tissue layer 120. Although embodiments of the present invention will be described with respect to human skin tissue 102, it will be understood that the present invention is applicable to creation of penetration in other types of tissue that are generally considered soft tissue other than bone, such as fascia, membranes, organs, vessels, vasculature, vascular pedicles, skin grafts, bladder, muscles, ligaments, tendons, as well as cartilage, and other biocompatible materials such as artificial skin, artificial membranes and synthetic mesh.

It has long been known that the most rapid healing of a skin opening 100 with a minimum of scarring occurs when vertical inner surfaces 108, 110 of the living dermal layer 118 at each side of the vertical interface 112 of skin opening 100 are brought together and held in close contact in what is referred to as an everted position as is shown in exaggerated fashion in FIG. 3. To the extent that the primarily non-living material of epidermal layer 114 can be excluded from the healing opening, the rapidity and level of scar tissue formed during the healing process will be minimized. One manner by which the epidermal layer 114 can be excluded from the healing opening is precisely control tissue capture and presentation prior to and during insertion of a surgical faster.

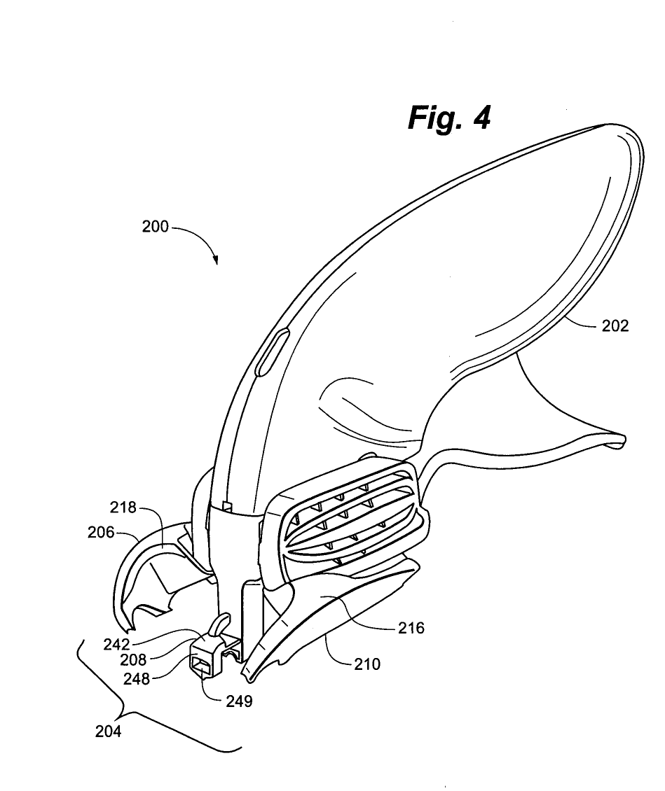

A representative surgical fastening apparatus 200 incorporating features of the present invention for precisely controlling tissue capture and presentation is illustrated generally in FIG. 4. Surgical fastening apparatus 200 can generally comprise a body 202 and a tissue interface portion 204. As depicted, body 202 may resemble the physical appearance of the aforementioned INSORB stapler although the tissue manipulation features of the various embodiments of the present invention are equally applicable to a variety of alternative body configurations including for example, traditional skin and laparoscopic stapling devices.

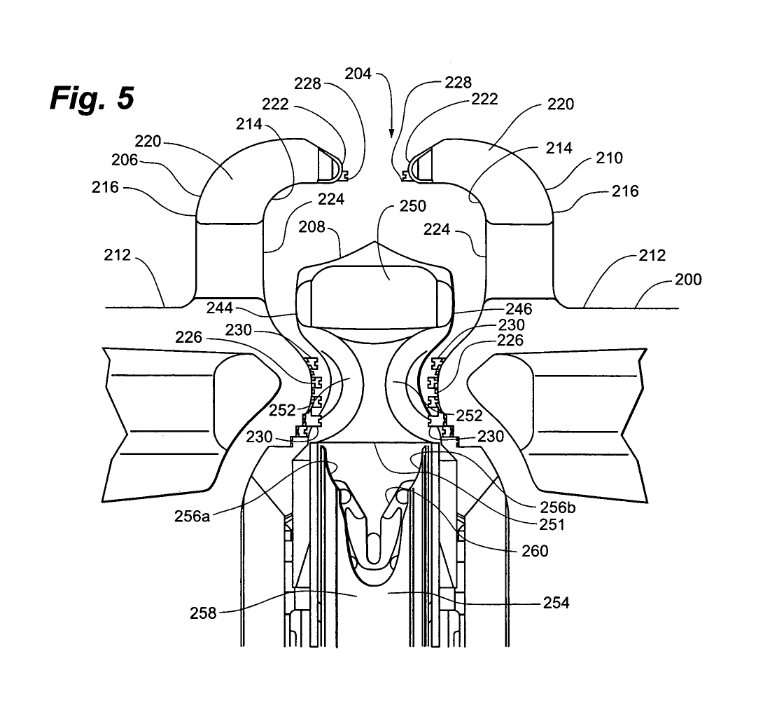

Tissue interface portion 204 is more clearly illustrated in FIG. 5. Tissue interface portion 204 can include a first capture arm 206, an insertion head 208 and a second capture arm 210. As depicted, first capture arm 206 and second capture arm 210 can be simultaneously, proximally located on opposed sides to the insertion head 208 though it is to be understood that variations on the surgical fastening apparatus 200 and fasteners could allow for and/or require sequential approximation of the first capture arm 206 and the second capture arm 210. Furthermore, the concepts related to tissue capture and positioning during insertion of a fastener can apply equally to instruments utilizing only a single capture arm, a single penetrator, a single curved penetrator and combinations thereof.

Referring to FIG. 5, first capture arm 206 and second capture arm 210 can be configured in a mirror image relation generally comprising an arm body 212 having a grasping surface 214, an exterior surface 216, a top surface 218 and a bottom surface 220. Grasping surface 214 can comprise a distal tip 222, a recessed surface 224 and a capture lobe 226. Distal tip 222 can comprise a distal grabbing member 228 while capture lobe 226 comprises a plurality of proximal gripping members 230. Distal gripping member 228 and proximal gripping members 230 may comprise either similar or dissimilar physical configurations.

Referring again to FIGS. 4 and 5, insertion head 208 can comprise a top head surface 242, a first side interface surface 244, a second side interface surface 246, an end surface 248, a penetrator bore 24, a bottom head surface 250 and a body interface portion 251. Both first side interface surface 244 and second side interface surface 246 can include a concave positioning guide 252.

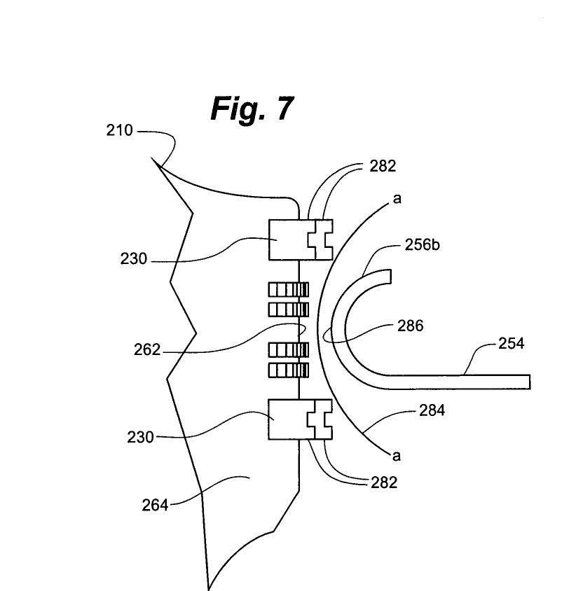

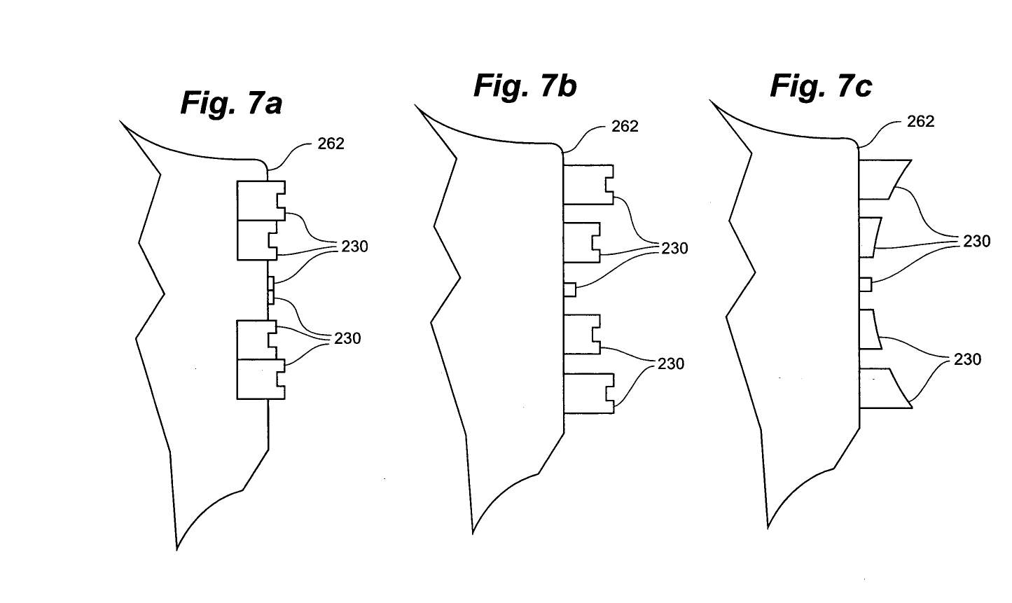

As illustrated in FIG. 5, surgical fastening apparatus 200 can comprise a penetrator assembly 254 located proximate the tissue interface portion 204. The penetrator assembly 254 can comprise a pair of penetrators 256a, 256b operably connected with a penetrator body member 258. While illustrated as having two penetrators 256a, 256b, it will be understood that the principles of tissue capture, maintenance and fastening are equally applicable to the use of a single penetrator. As illustrated in FIG. 7, penetrator assembly 254 can be utilized to carry and deposit a surgical fastener 260 as will be further described below.

Surgical fastener 260 can comprise suitable materials of construction such as, for example, biologically compatible metals such as stainless steel or nitinol or biologically compatible plastics. In one embodiment, surgical fastener 260 can comprise a bioabsorbable fastener as described in U.S. Pat. No. 7,112,214, which is herein incorporated by reference in its entirety. It will be recognized that in alternate embodiments, a surgical apparatus in accordance with the present invention may be utilized for creating controlled penetrations of tissue for a variety of surgical purposes, including insertion of fasteners, such as for wound closure, tissue anchoring, stabilization, or repair, as well as attachment or anchoring of medical devices, such as slings, mesh, or implantable apparatus, or medical implants, such as ligaments, tendons, muscles, or organs.

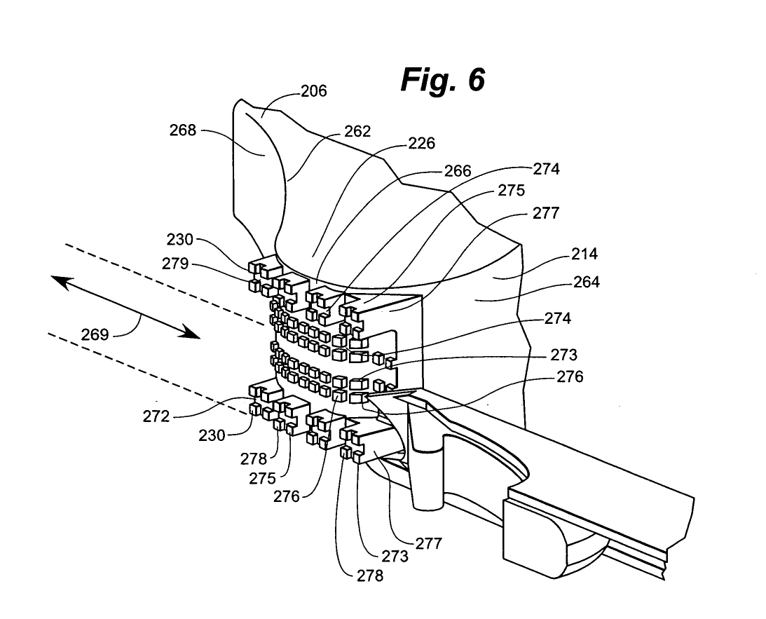

Capture lobe 226 is more clearly illustrated in FIG. 6. Capture lobe 226 generally comprises a continuous lobe surface 262 defined by a lobe leading surface 264, a lobe interface surface 266 and a lobe trailing surface 268. In one representative embodiment, continuous lobe surface 262 has a generally convex appearance. Proximal gripping members 230 are arranged in both a first direction and a second direction along the continuous lobe surface 262, wherein the first direction is generally parallel to and along a penetrator travel path 269 while a second direction is generally transverse to the first direction Proximal gripping members 230 define a capture surface 272 having a plurality of raised edges 273 and a plurality of channels 274. Raised edges 273 present gripping edges for gripping tissue while channels 274 proved spaces into which tissue can be compressed and/or squeezed. Raised edges 273 and channels 274 can be arranged in a variety of orientations, for example, along the first direction, the second direction and combinations thereof. Capture surface 272 can be presented using a variety of configurations for proximal gripping members 230 including, for example, a raised castle configuration 275 or a raised cube 276. Raised castle configuration 275 can comprise a projecting body 277 in which, capture surface 272 is generally defined by a plurality of projecting corner cubes 278 extending upward from a surface 279 of the projecting body 277. Either alternatively or in conjunction with raised castle configuration 275, a plurality of raised cubes 276 can be arranged along the continuous lobe surface 262, and more specifically the lobe interface surface 266 so as to define the capture surface 272. While gripping members 230 are described and illustrated as comprising cube or castle configurations, it will be understood that a variety of geometrical configurations can be utilized to present raised edges and channels including, for example, cylinders, pyramids, crosses and the like. In addition, surface 279 on a projecting body for any suitable geometric configuration of gripping member 230 can comprise a concave or scallop-shaped surface.

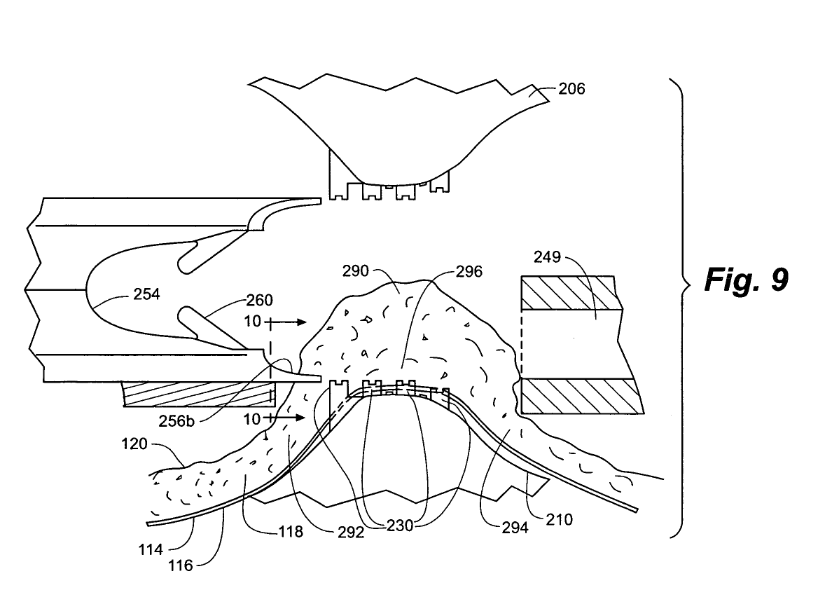

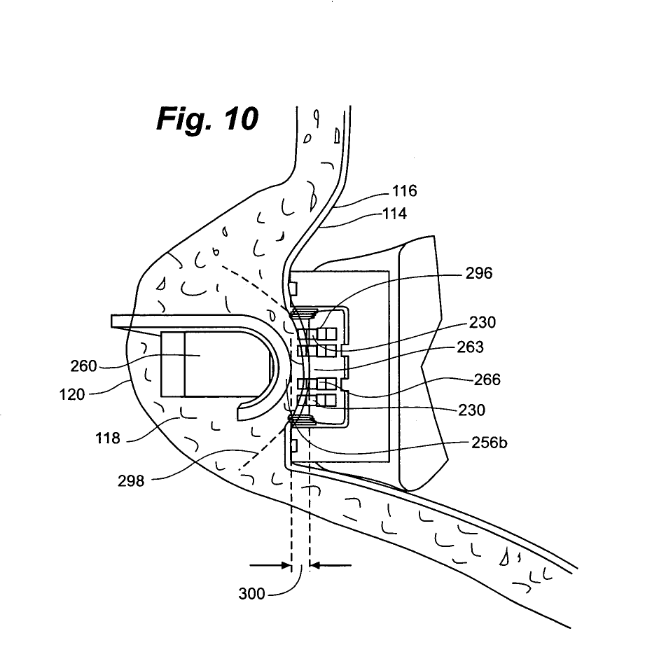

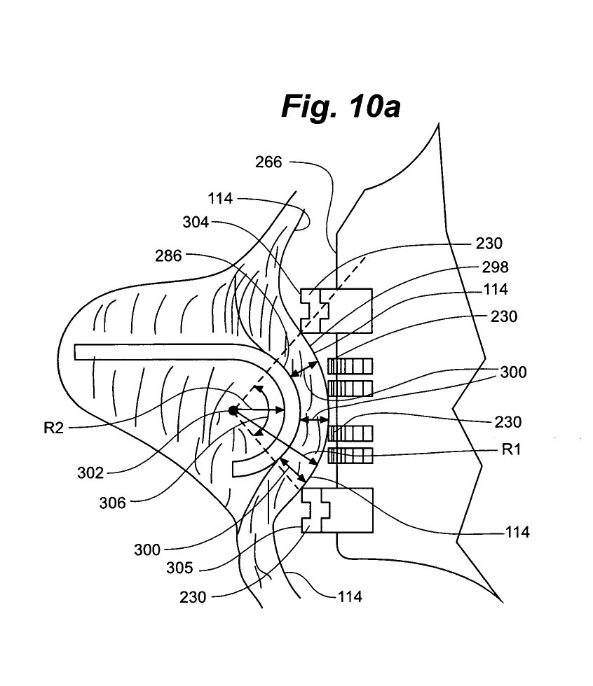

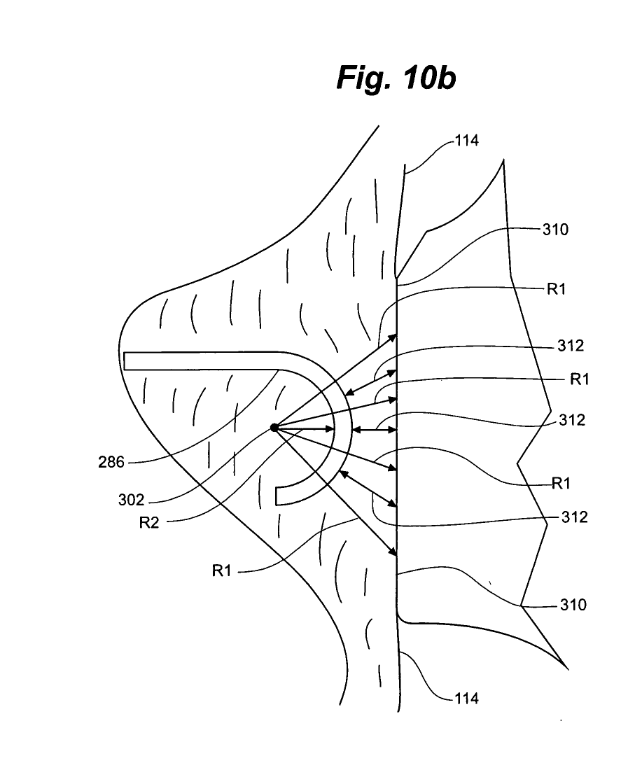

Proximal gripping members 230 are arranged in the second direction in a staggered configuration 282 as illustrated in FIG. 7 such that tissue can be captured, retained and positioned at varying distances from the continuous lobe surface 262 so as to define an arcuate gripping radius 284 illustrated at arc a-a in FIG. 7 wherein at least a portion of arcuate gripping radius 284 corresponds to a penetrator piercing arc 286. As will be illustrated and described below, the similarity between arcuate gripping arc 284 and penetrator piercing arc 286 allows for piercing of tissue and corresponding placement of surgical fastener 260 at a fixed placement distance or depth 288 within the dermal layer 118 as measured from the exterior surface 118 of epidermal layer 114 as illustrated in FIGS. 9 and 10. Proximal gripping members 230 can be arranged along continuous lobe surface 262 in any of a variety of suitable configurations for defining arcuate grip radius 284 as illustrated in FIGS. 7a, 7b and 7c.

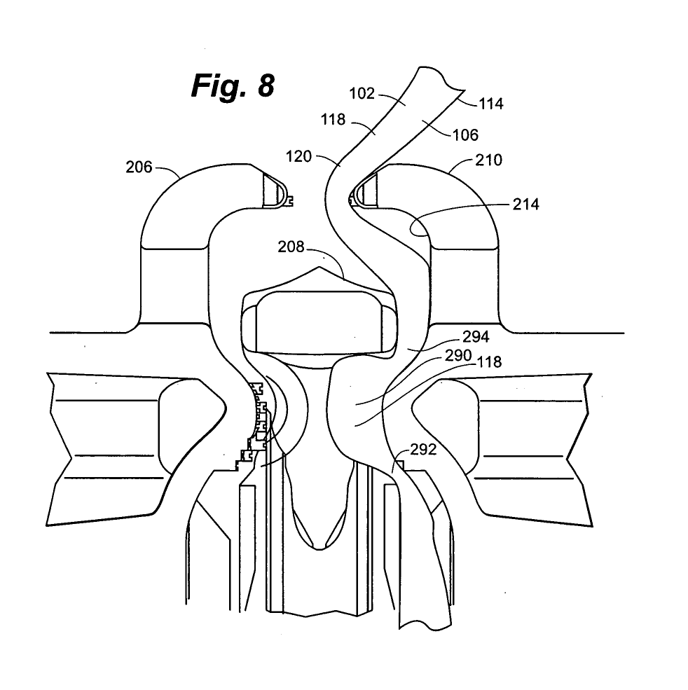

Use of surgical fastening apparatus 200 and the advantages of the tissue capture and maintenance components described above is illustrated in FIGS. 8, 9, 10, 11 and 12. For purposes of clarity and to better describe the use of surgical fastening apparatus 200, many of the drawings are illustrated from a bottom viewing orientation in which second side 106 is illustrated being pierced and captured while first side 104 is not depicted so as to more clearly illustrate the interactions occurring at tissue interface portion 204. It will be understood that in certain representative embodiments, first side 104 and second side 106 can be either simultaneously or sequentially captured at tissue interface portion 204.

As illustrated in FIGS. 9 and 10, a first tissue capture step is illustrated in which surgical fastening apparatus 200, and more specifically, tissue interface portion 204 has captured second side 106 between grasping surface 214 on second capture arm 210 and insertion head 208 using the actuation mechanisms and procedures described in U.S. Patent Publication 20040059377, which is herein incorporated by reference in its entirety. As seen in FIG. 8, a clearance space 290 between lobe interface surface 266 and concave positioning guide 252 is generally increased in comparison to a leading clearance space 292 between the concave positioning guide 252 and the lobe leading surface 264 and a trailing clearance space 294 between the concave positioning guide 252 and the lobe trailing surface 268. Due to the viscoelastic nature of skin 102, and most especially dermal layer 118 and subcutaneous tissue layer 120, clearance space 290 allows increased amounts of tissue to be squeezed out of leading clearance space 292 and trailing clearance space 294. This squeezing action at leading clearance space 292 and trailing clearance space 294 creates tension along the length of the skin 102 captured in clearance space 290 such that a tissue drape 296 is formed along the lobe interface surface 264 that substantially assumes a tissue drape arc 298 substantially resembling arcuate gripping radius 284 as illustrated in FIG. 7.

As tissue drape 296 is formed along lobe interface surface 266, the epidermal layer 114 is stretched across and squeezed into the proximal gripping members 230. Regardless of how capture surface 272 is defined, for example with raised castle configuration 275 or raised cube 276 and combinations thereof, the combination of raised edges 273 and channels 274 of capture surface 272 grips the epidermal layer 114 and provides traction that prevents the epidermal layer 114 from sliding along the lobe interface surface 266 as well as maintaining the arcuate nature of tissue drape arc 298. The combination of raised edges 273 and channels 274 provides omni-directional support on tissue that is captured and squeezed against the proximal gripping members so as to avoid slippage of the captured tissue along either the first direction, the second direction or combinations of directions relative to the lobe interface surface 266. Proximal gripping members 230 can be especially beneficial in gripping epidermal layer 114 in instances when the epidermal layer is wet and/or lubricated with anesthetics, lotions or other topicals. In addition, the tractive features provided by proximal gripping members 230 can serve to isolate the captured tissue from external user based forces such as operator fatigue or ergonomic issues that could negatively impact the tissue capture and subsequent tissue fastening.

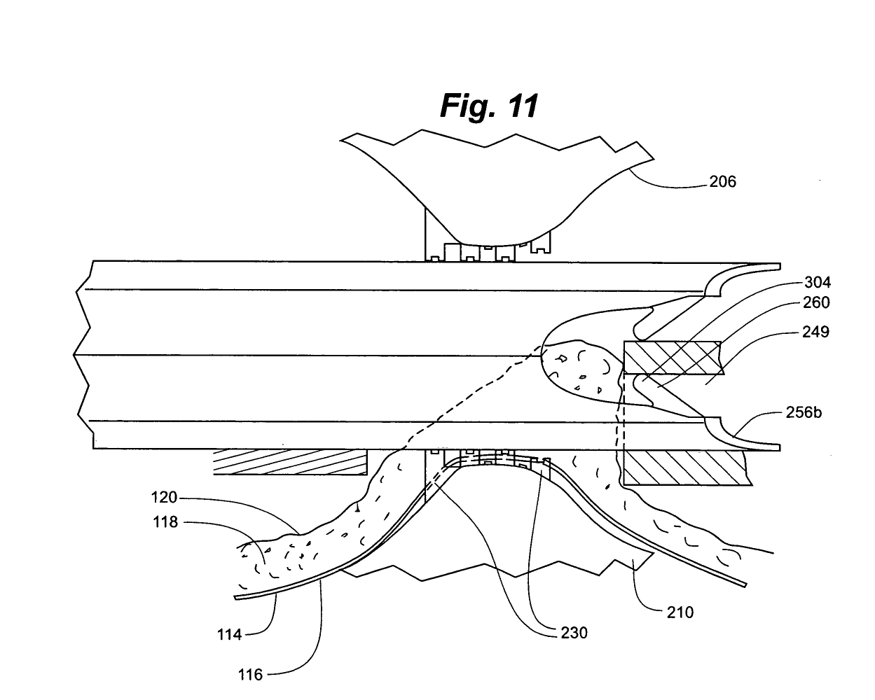

As penetrator 256b is advanced into and through the tissue within clearance space 290, dermal layer 118 and subcutaneous tissue layer 120 essentially piles up against the penetrator bore 249 as illustrated in FIG. 11, while the proximal gripping members 230 maintain the position of epidermal layer 114 and counteract the force of penetration. As the penetrator 256b approaches penetrator bore 249, penetrator 256b pierces the dermal layer 118 of tissue drape 296. As the penetrator 256b pierces the dermal layer 118, the tissue drape arc 298 insures that penetrator 256b does not pierce or button-hole the epidermal layer 114 by maintaining an essentially constant insertion depth 300 shown in FIG. 10 measured from exterior surface 116 of skin 102. Generally, constant insertion depth 300 is within a range of about 0.005″ to about 0.021″, and is most preferably about 0.013″, such that penetrator 256b is always piercing within the dermal layer 118. As constant insertion depth 300 is physically defined by the interaction between the penetrator 256b and the lobe interface surface 266/proximal gripping members 230, the constant insertion depth 230 can be maintained regardless of differing user techniques, operator fatigue or tissue variability that is often encountered during closure of large incisions. As penetrator 256b enters the penetrator bore 249, the tissue piled up against the penetrator bore 249 is pulled over a retention member illustrated as cleat 304 on the surgical fastener 260 such that surgical fastener 260 resides in the hole cut by penetrator 256b in dermal layer 118.

The principles for consistently defining constant insertion depth 300 are further illustrated in FIGS. 10a and 10b. Referring to FIG. 10a, proximal gripping members 230 along lobe interface surface 266 grip the epidermal layer 114 to define the tissue drape arc 298. The tissue drape arc 298 has a generally constant radius R1 centered upon a penetrator center point 302. Likewise, the penetrator piercing arc 286 has a generally constant radius R2 centered upon the penetrator center point 302. Tissue drape arc 298 remains generally constant between an uppermost proximal gripping members 304 and lowermost proximal gripping members 305 such that R1 is constant for an arc length 306 of about 60° to about 120°, and most preferably greater than about 90°.

Referring to FIG. 10b, a prior art lobe interface surface 310 is illustrated that lacks proximal gripping members 230, and thus has no mechanism for defining tissue drape arc 298. While, the penetrator piercing arc 286 has continues to have generally constant radius R2 centered upon the penetrator center point 302, radius R1 is no longer constant such that a variable insertion depth 312 is depicted.

One advantage of the physical definition of constant insertion depth 300 provided by the present disclosure is that it allows for two medical professionals to work from opposite ends of large incisions so as to reduce overall closure time, while providing consistent placement of surgical fastener 260 regardless of operator technique or fatigue. This can be especially valuable in especially large closure situations such as, for example, an abdominoplasty procedure.

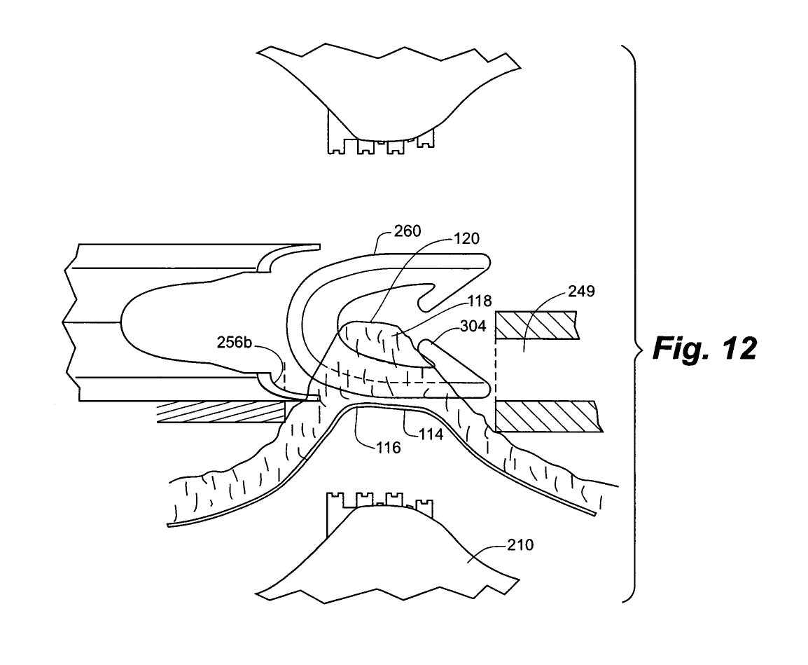

Once penetrator 256b has carried the surgical fastener 260 into the penetrator bore 249, the penetrator assembly 254 can be withdrawn as illustrate in FIG. 12 such that penetrator 256b backs out of the hole pierced in dermal layer 118. As penetrator assembly 254 is withdrawn, the tissue pulled over cleat 304 prevents the surgical fastener 260 from being withdrawn. As penetrator assembly 254 is fully withdrawn, second capture arm 210 can be withdrawn from insertion head 208 thereby releasing the captured tissue and allowing for tissue interface portion 204 to be removed or repositioned for subsequent tissue capture and fastener placement along the length of opening 100.

While representative embodiments of the invention have been described with respect to fastening of skin, and more particularly, dermal tissue, it will be recognized that the invention is also applicable to other types of tissue such as facia, muscle, ligaments, cartilage, tendons and the like.

Although the invention has been described with respect to a variety of representative embodiments, it will be understood that numerous insubstantial changes in configuration, arrangement or appearance of the elements of the present invention can be made without departing from the intended scope of the invention. Accordingly, it is intended that the scope of the invention be determined by the claims as set forth.

What is claimed:

1. A surgical apparatus comprising:
a body assembly including a penetrator assembly having a penetrator movable along a penetrator pathway; and
a tissue interface portion, the tissue interface portion having a insertion head and a at least one tissue capture arm, each capture arm having a convex capture lobe for interfacing with a concave positioning guide on the insertion head and wherein each convex capture lobe includes a plurality of gripping members arranged along a lobe interface surface of the convex capture lobe in a first direction generally parallel to and along the penetrator path and in a second direction generally transverse to the first direction, wherein an arrangement of the plurality of gripping members in the second direction defines an arcuate gripping radius,
such that a tissue drape formed of tissue captured between the convex capture lobe and the concave positioning guide has a tissue drape arc that generally conforms to the arcuate gripping radius and the arcuate gripping radius defines a substantially constant insertion depth of tissue relative to a surface of the penetrator along at least a portion of the penetrator pathway.

2. The surgical apparatus of claim 1, wherein the constant insertion depth is between about 0.005 inches to about 0.021 inches.

3. The surgical apparatus of claim 1, wherein the plurality of gripping members define a capture surface including a plurality of raised edges and a plurality of channels for gripping a surface of tissue.

4. The surgical apparatus of claim 3, wherein the gripping members can comprise a raised castle configuration, a raised cube or combinations thereof.

5. The surgical apparatus of claim 1, wherein an uppermost gripping member and a lowermost gripping member on the lobe interface surface relative to the second direction define endpoints of the tissue drape arc such that the tissue drape arc extends between about 60° to about 120°.

6. The surgical apparatus of claim 5, wherein the tissue drape arch exceeds at least about 90°.

7. The surgical apparatus of claim 1, wherein the convex capture lobe further includes a lobe leading surface and a lobe trailing surface, with a clearance space defined between the lobe interface surface and the concave positioning guide that exceeds a leading clearance space defined between the lobe leading surface and the concave positioning guide and a trailing clearance space defined between the lobe trailing surface and the concave positioning guide, wherein tissue captured between the insertion head and the at least one capture arm is squeezed out of the leading clearance space and the trailing clearance space and into the clearances space creating tension in the captured tissue leading to formation of the tissue drape along the lobe interface surface.

8. A method for penetrating soft tissue other than bone in a patient, comprising:
providing a surgical apparatus having a penetrator movable along a penetrator pathway in a first direction, the surgical apparatus including a capture arm and an insertion head;
grasping tissue between the capture arm and the insertion head to form a tissue drape;
forming a tissue drape arc along an interior surface of the capture arm in a second direction generally transverse to the first direction, the interior surface including an arrangement of a plurality of gripping members that defines an arcuate gripping radius in the second direction; and
advancing the penetrator through the tissue drape arc in the first direction such that the penetrator cuts through the tissue drape arc at a substantially constant insertion depth relative to a surface of the penetrator along at least a portion of the penetrator pathway.

9. The method of claim 8, further comprising:
carrying a fastener with the penetrator such that the fastener can be positioned within the tissue as the substantially constant insertion depth.

10. A method for isolating external forces during creation of a penetration in soft tissue other than bone in a patient, comprising:
providing a surgical apparatus having at least one capture arm and an insertion head adapted to capture tissue therebetween, the at least one capture arm including a plurality of tissue gripping members defined along an interface surface of the capture arm and the insertion head including a penetrator;
grasping tissue between the capture arm and the insertion head to form a tissue drape such that horizontal slippage of the tissue drape is controlled by grasping an external surface of the tissue drape with the tissue gripping members; and
presenting a generally constant insertion depth of the tissue drape to the penetrator by forming a tissue drape arc conforming to a gripping radius defined by the plurality of tissue gripping members.

11. The method of claim 10 further comprising:
advancing the penetrator through the tissue drape arc to create the penetration in soft tissue.

12. The method of claim 11, further comprising:
placing a surgical fastener in at least a portion of the penetration in soft tissue.

13. The method of claim 10, wherein grasping tissue comprises:
squeezing tissue out of a leading clearance space and a trailing clearance space and into a clearance space as defined by the insertion head proximate an interface surface of the penetrator such that tension is placed on tissue along the interface surface to define the tissue drape.

14. A method for providing instruments and instructions for penetrating soft tissue other than bone in a patient, comprising:
providing a surgical apparatus having a penetrator movable along a penetrator pathway in a first direction, the surgical apparatus including a capture arm and an insertion head;
providing instructions for operating the surgical apparatus by:
grasping tissue between the capture arm and the insertion head to form a tissue drape;
forming a tissue drape arc along an interior surface of the capture arm in a second direction generally transverse to the first direction, the interior surface including an arrangement of a plurality of gripping members that defines an arcuate gripping radius in the second direction; and
advancing the penetrator through the tissue drape arc in the first direction such that the penetrator cuts through the tissue drape arc at a substantially constant insertion depth relative to a surface of the penetrator along at least a portion of the penetrator pathway.

15. The method of claim 14, wherein the surgical apparatus includes at least one fastener and advancing the penetrator further comprises:
carrying a fastener with the penetrator such that the fastener can be positioned within the tissue as the substantially constant insertion depth.
